SEQUENCE LISTING (23 sequences)

SEQ ID NO: 1 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 2 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 3 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 4 - protein, 379 aa, Clostridium acetobutylicum
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 5 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 6 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 7 - protein, 336 aa, Clostridium acetobutylicum
MNKADYKGVWVFAEQRDGELQKVSLELLGKGKEMAEKLGVELTAVLLGHNTEKMSKDLLSHGADKVLAADNELLAHFSTDGYAKVICDLVNERKPEILFIGATFIGRDLGPRIAARLSTGLTADCTSLDIDVENRDLLATRPAFGGNLIATIVCSDHRPQMATVRPGVFEKLPVNDANVSDDKIEKVAIKLTASDIRTKVSKVVKLAKDIADIGEAKVLVAGGRGVGSKENFEKLEELASLLGGTIAASRAAIEKEWVDKDLQVGQTGKTVRPTLYIACGISGAIQHLAGMQDSDYIIAINKDVEAPIMKVADLAIVGDVNKVVPELIAQVKAANN

SEQ ID NO: 8 - DNA, 1011 bp, Artificial sequence
  ETF alpha codon pair optimised
atgaacaaggctgactacaagggtgtctgggttttcgctgaacaaagagatggtgaattacaaaaggtttctttggaattgctaggtaagggtaaggaaatggctgaaaaattgggtgttgaattgactgctgtcttattgggtcacaacactgaaaagatgtccaaggacttgttgtcccacggtgctgacaaggttttggctgctgacaacgaattattagctcatttctccactgacggttacgccaaggttatctgtgacttggtcaacgaaagaaagccagaaatcttattcatcggtgctactttcatcggtagagacttgggtccaagaattgctgccagattgtctactggtttgactgctgactgtacctctttggatatcgatgtcgaaaaccgtgacttgttggctaccagaccagctttcggtggtaacttgattgctaccattgtctgttctgaccacagacctcaaatggccaccgtcagaccaggtgtctttgaaaaattgccagttaacgatgctaacgtttctgatgacaagatcgaaaaggttgccatcaaattgactgcttctgatatcagaaccaaggtttccaaggttgtcaaattggccaaggacattgctgatatcggtgaagccaaggttttggttgctggtggtcgtggtgttggttccaaggaaaacttcgaaaaattggaagaattagcttctttgttgggtggtaccattgctgcttccagagctgccattgaaaaggaatgggttgacaaggacttgcaagtcggtcaaaccggtaagaccgtcagaccaactttgtacattgcttgtggtatctctggtgccatccaacacttggctggtatgcaagactctgactacatcattgccatcaacaaagatgtcgaagctccaatcatgaaggttgctgatttggccattgtcggtgatgtcaacaaggttgttccagaattgattgctcaagtcaaggctgctaacaattaa

SEQ ID NO: 9 - protein, 259 aa, Clostridium acetobutylicum
MNIVVCLKQVPDTAEVRIDPVKGTLIREGVPSIINPDDKNALEEALVLKDNYGAHVTVISMGPPQAKNALVEALAMGADEAVLLTDRAFGGADTLATSHTIAAGIKKLKYDIVFAGRQAIDGDTAQVGPEIAEHLGIPQVTYVEKVEVDGDTLKIRKAWEDGYEVVEVKTPVLLTAIKELNVPRYMSVEKIFGAFDKEVKMWTADDIDVDKANLGLKGSPTKVKKSSTKEVKGQGEVIDKPVKEAAAYVVSKLKEEHYI

SEQ ID NO: 10 - DNA, 780 bp, Artificial sequence
  ETF beta codon pair optimised
atgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaa

SEQ ID NO: 11 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 12 - DNA, 1407 bp, Artificial sequence
  acdh L. innocua cpo
atggaatctttggaattggaacaattagtcaagaaggttttgttggaaaaattggctgaacaaaaggaagttccaaccaagaccaccacccaaggtgccaagtccggtgttttcgataccgtcgatgaagctgtccaagctgccgtcattgctcaaaactgttacaaggaaaaatctttggaagaaagaagaaacgttgtcaaggccatcagagaagctttatacccagaaatcgaaaccattgctaccagagctgttgctgaaaccggtatgggtaatgtcaccgataaaatcttgaagaacactttagctatcgaaaagactccaggtgttgaagacttgtacactgaagttgctaccggtgacaacggtatgactttatacgaattatctccatacggtgtcatcggtgctgttgctccatctaccaacccaactgaaactttgatctgtaactccatcggtatgttggctgctggtaacgccgttttctactctcctcacccaggtgccaagaacatctctttatggttgattgaaaagttgaacactatcgtcagagattcttgtggtattgacaacttgattgtcaccgttgccaagccatctatccaagctgctcaagaaatgatgaaccacccaaaggttccattgttggtcatcactggtggtccaggtgttgtcttgcaagctatgcaatctggtaagaaggttatcggtgctggtgctggtaaccctccatccatcgttgacgaaaccgctaacattgaaaaggctgctgctgacattgtcgacggtgcttcctttgaccataatatcttgtgtatcgctgaaaagtctgttgttgccgttgactccattgctgacttcttgttgttccaaatggaaaagaacggtgctttgcacgtcactaacccatctgatatccaaaaattggaaaaggttgccgtcactgacaagggtgtcaccaacaagaaattggttggtaagtctgccactgaaatcttgaaagaagctggtattgcttgtgatttcaccccaagattgatcattgtcgaaactgaaaagtcccacccattcgctactgttgaattgttgatgccaattgttccagttgtcagagttccagacttcgatgaagctttggaagttgccattgaattggaacaaggtctacatcacactgctaccatgcactctcaaaacatctccagattgaacaaggctgcccgtgacatgcaaacctccatctttgtcaagaacggtccatctttcgctggtttaggtttcagaggtgaaggttccaccactttcaccattgctactccaactggtgaaggtactaccactgcccgtcacttcgctagaagaagaagatgtgtcttgactgatggtttctccattaga

SEQ ID NO: 13 - DNA, 13286 bp, Artificial sequence
  pBOL34 integration vetcor
aagcttgcatgcctgcaggtcgacggcgcgccgggcccgtttaaacggccggccaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgaaggaagttgttattgcttctgctgtcagaactgccattggttcttacggtaagtctttgaaggacgtcccagctgtcgacttgggtgctaccgccatcaaggaagctgtcaagaaggctggtatcaagccagaagatgttaacgaagttatcttaggtaacgttttgcaagctggtttaggtcaaaacccagctcgtcaagcttctttcaaggctggtttgccagttgaaattccagccatgaccatcaacaaggtttgtggttctggtttgagaactgtttctttggctgctcaaatcatcaaggctggtgacgctgatgtcatcattgctggtggtatggaaaacatgtccagagctccatacttggctaacaatgctagatggggttacagaatgggtaacgccaagttcgtcgatgaaatgatcactgacggtttatgggacgctttcaacgactaccacatgggtatcactgctgaaaacattgctgaaagatggaacatctccagagaagaacaagatgaatttgctttggcttctcaaaagaaggctgaagaagccatcaaatctggtcaattcaaggacgaaattgtcccagttgtcatcaagggtagaaagggtgaaaccgttgtcgacaccgatgaacacccaagattcggttccaccattgaaggtttggccaagttgaaaccagctttcaagaaggatggtaccgtcactgctggtaacgcttccggtttgaacgactgtgctgctgttttggttatcatgtctgctgaaaaggccaaggaattgggtgtcaagccattggccaagattgtctcctacggttctgctggtgttgacccagccatcatgggttacggtcctttctacgctaccaaggctgctatcgaaaaggctggttggaccgttgacgaattggatttgattgaatccaacgaagctttcgctgctcaatctttggctgttgccaaggacttgaaattcgacatgaacaaggtcaacgttaacggtggtgccattgctttgggtcacccaattggtgcttccggtgccagaatcttggttactttagtccacgctatgcaaaagcgtgatgccaagaagggtttggctactctatgtatcggtggtggtcaaggtactgccatcttattggaaaagtgttaggcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaagtttaaacatttaaacgtgtgtgtgcattatatatattaaaaattaagaattagactaaataaagtgtttctaaaaaaatattaaagttgaaatgtgcgtgttgtgaattgtgctctattagaataattatgacttgtgtgcgtttcatattttaaaataggaaataaccaagaaagaaaaagtaccatccagagaaaccaattatatcaaatcaaataaaacaaccagcttcggtgtgtgtgtgtgtgtgaagctaagagttgatgccatttaatctaaaaattttaaggtgtgtgtgtggataaaatattagaatgacaattcgagatgaaattttaagcaaactctagtaggaaataagcggcttattcttgttggctcctaattctttttagtgtatcagttcccattgataaaaaaattaaaattaaaattagaaaaattaaaccagaaaaatcaagttgattaaaatgtgacaaaaattatgattaaatgctacttcaacaggagcccgggcctatttggagtagtcgtagaaacccttaccagactttctacctaaccaaccagctctaacgtacttcttcaataaagtgtgaggtctgtacttagagtcaccggtttcagagtataagacatccatgatggccaaacagatatccaaaccgatgaagtcacctaattccaatggacccattgggtggttagcacccaatttcatggccttgtcgatatcttcaacagaagcaataccttcagccaaaataccgacagcttcgttgatcattggaatcaagattctgttgacaacgaaacctggagcttcagcaacttcaactgggtccttaccaatggcaatggaagtttccttgacagcatcgaaagtttcttgagaggtggcaatacctctgatgacttcgaccaacttcatgactggagctgggttgaagaagtgcataccgataaccttgtctggtctcttggtagcagaagcaacttcagtgatggacaaagaagaagtgttggaagccaaaatggtttctggcttacagatgttgtccaaatcagcaaagatttgcttcttgatgtccattctttcaacggcagcttcaatgaccaaatcacagtcagcagccatgttcaagtcaacagtaccggagattctggtcaagatttcgaccttggtagcttcttcaatcttacccttcttgaccaacttggacaagttcttgttgatgaaatccaaaccacggtcaacgaattcgtccttgatatctctcaaaacaacttcgaaacccttggcagcgaaagcttgagcaataccagaacccatggtaccggcaccaatgacacaaaccttcttcattttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagcggtatttttctttttttttcttccttctttttcgtcattatttcaaacgcttttgcgtcaagtaatgaatatctggcggttccgcggtaatgcgacaatttgtgatatgcactcttaaaaccccgccacgatgatcgcacgtgccggcatttatagacgacttttctggttgtcccgcttcacggcacatgcatgcatcaatgaccgaattcaggttgctactaaccattgtgttgtgttattgctgtgcatgaggtgctcaagtgcccgcggcatctgactagtggtaactctagacggcttcgatgcagagagttcctcaaaatttttcttttcaattgtttgcctggtttccgcggcgtatatcagtttttggcgatatggtaacgcgatactctacggcaccttcacggtagatgtcttttttaaaagtgactgttaattccaggattgaaaggaagtgtcgaatagtatagtatgctttctaggccggccgtttaaatgggcccgcggcccgtttaaacggccggcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacataacaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatggaattgaacaacgttatcttggaaaaggaaggtaaggttgccgttgtcaccatcaacagaccaaaggctttgaatgctttgaactctgacactttgaaggaaatggactacgtcattggtgaaattgaaaacgattctgaagttttggctgtcatcttgaccggtgccggtgaaaagtctttcgttgctggtgctgatatctctgaaatgaaggaaatgaacaccattgaaggtagaaagttcggtatcttaggtaacaaggttttcagaagattggaattgttggaaaagccagtcattgctgctgtcaacggtttcgctttgggtggtggttgtgaaattgccatgtcctgtgacatcagaattgcttcttctaacgctcgtttcggtcaaccagaagtcggtctaggtatcactccaggtttcggtggtactcaaagattatccagattggttggtatgggtatggccaagcaattgatcttcaccgctcaaaacatcaaggctgacgaagctttgagaattggtttagtcaacaaggttgttgaaccatctgaattgatgaacactgccaaggaaattgctaacaagatcgtctccaacgctccagttgctgtcaaattgtccaagcaagccatcaacagaggtatgcaatgtgatatcgacaccgctttggcctttgaatctgaagctttcggtgaatgtttctccactgaagaccaaaaggatgctatgaccgctttcatcgaaaagagaaagattgaaggtttcaagaacaggtgatgagcccgggcgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatccacagcctagccttcagttgggctctatcttcatcgtcattcattgcatctactagccccttacctgagcttcaagacgttatatcgcttttatgtatcatgatcttatcttgagatatgaatacataaatatatttactcaagtgtatacgtgcatgctttttttacggtttaaacatttaaatgggccgctctagaggatccccgggtaccgagctcgggcccagcgctactagttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagagacccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtcgagaatttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatcaaagtaaaaaaaagattttcgctacatgttccttttgaagaaagaaaatcatggaacattagatttacaaaaatttaaccaccgctgattaacgattagaccgttaagcgcacaacaggttattagtacagagaaagcattctgtggtgttgccccggactttcttttgcgacataggtaaatcgaataccatcatactatcttttccaatgactccctaaagaaagactcttcttcgatgttgtatacgttggagcatagggcaagaattgtggcttgagatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 14 - DNA, 16359 bp, Artificial sequence
  pBOL36 integration vector
aagcttgcatgcctgcaggtcgacggcgcgccgggcccgtttaaacaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtgggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacaggccggccaaaatgaaggccaaatcaaggcgggaagggacaaccaggacgtaaagggtagcctccccataacataaactcaataaaatatatagtcttcaacttgaaaaaggaacaagctcatgcaaagaggtggtacccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattatcttctactcataacctcacgcaaaataacacagtcaaatcaatcaaaatggacttcaacttgaccagagaacaagaattggtcagacaaatggttagagaatttgctgaaaacgaagttaagccaattgctgctgaaatcgatgaaactgaaagattcccaatggaaaacgtcaagaagatgggtcaatacggtatgatgggtattccattctctaaggaatacggtggtgctggtggtgacgtcttgtcttacatcattgctgtcgaagaattgtccaaggtttgtggtaccactggtgtcatcttatctgctcacacttctctatgtgcctccttgatcaacgaacacggtactgaagaacaaaagcaaaagtacttggttccattggccaagggtgaaaagattggtgcctacggtttgactgaaccaaacgctggtactgactctggtgctcaacaaactgttgccgttttggaaggtgaccactacgtcatcaacggttccaagatcttcatcaccaacggtggtgttgctgacacctttgtcatcttcgctatgaccgatcgtaccaagggtaccaagggtatctctgctttcattattgaaaagggtttcaagggtttctccatcggtaaggtcgaacaaaagttgggtatcagagcttcctctaccactgaattggttttcgaagacatgattgttccagttgaaaacatgatcggtaaggaaggtaagggtttcccaattgccatgaagactttagatggtggtagaattggtattgctgctcaagctttgggtattgctgaaggtgccttcaacgaagctagagcttacatgaaggaaagaaagcaattcggtagatctttggacaaattccaaggtttggcttggatgatggctgacatggacgttgccatcgaatctgctcgttacttggtctacaaggctgcttacttgaagcaagctggtttgccatacaccgtcgatgctgccagagctaagttgcacgctgccaacgttgccatggatgtcaccaccaaggctgtccaattattcggtggttacggttacaccaaggactacccagttgaaagaatgatgagagatgctaagatcactgaaatctacgaaggtacttctgaagttcaaaagttggttatctccggtaagatcttcagataggcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaagtttaaacatttaaacggccggcctagaaagcatactatactattcgacacttcctttcaatcctggaattaacagtcacttttaaaaaagacatctaccgtgaaggtgccgtagagtatcgcgttaccatatcgccaaaaactgatatacgccgcggaaaccaggcaaacaattgaaaagaaaaattttgaggaactctctgcatcgaagccgtctagagttaccactagtcagatgccgcgggcacttgagcacctcatgcacagcaataacacaacacaatggttagtagcaacctgaattcggtcattgatgcatgcatgtgccgtgaagcgggacaaccagaaaagtcgtctataaatgccggcacgtgcgatcatcgtggcggggttttaagagtgcatatcacaaattgtcgcattaccgcggaaccgccagatattcattacttgacgcaaaagcgtttgaaataatgacgaaaaagaaggaagaaaaaaaaagaaaaataccgcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaaatggtcgatttcgaatactctatcccaaccagaatcttcttcggtaaggacaagatcaacgttttgggtagagaattgaagaaatacggttccaaggttttgattgtctacggtggtggttccatcaagagaaacggtatctacgacaaggctgtctccattttggaaaagaactctatcaaattctacgaattggctggtgttgaaccaaacccaagagttaccaccgtcgaaaagggtgtcaagatctgtcgtgaaaacggtgttgaagttgttttggccatcggtggtggttctgccattgactgtgccaaggtcattgctgctgcctgtgaatacgatggtaacccatgggacattgtcttggatggttctaagatcaagcgtgtcttaccaattgcttccatcttgactatcgctgctactggttctgaaatggacacctgggctgttatcaacaacatggacactaacgaaaagttgattgctgctcacccagatatggccccaaagttctctattttggacccaacctacacttacactgttccaaccaaccaaactgctgctggtactgctgatatcatgtctcacatctttgaagtttacttctccaacaccaagaccgcttacttgcaagacagaatggctgaagctctattaagaacctgtatcaagtacggtggtattgctttggaaaagccagatgactacgaagccagagctaacttgatgtgggcttcctctttggctatcaacggtttattgacttacggtaaggacaccaactggtccgttcatttgatggaacacgaattgtctgcttactacgatatcactcacggtgtcggtttggccatcttgactccaaactggatggaatacattttgaacaacgacactgtctacaagttcgtcgaatacggtgttaacgtctggggtattgacaaggaaaagaaccactacgacattgctcaccaagccatccaaaagaccagagactatttcgtcaacgttttgggtttaccatccagattaagagatgttggtattgaagaagaaaaattggatatcatggctaaggaatctgtcaaattgactggtggtaccattggtaacttgagacctgttaacgcttctgaagttttgcaaatcttcaagaaatctgtttaggcccgggctcctgttgaagtagcatttaatcataatttttgtcacattttaatcaacttgatttttctggtttaatttttctaattttaattttaatttttttatcaatgggaactgatacactaaaaagaattaggagccaacaagaataagccgcttatttcctactagagtttgcttaaaatttcatctcgaattgtcattctaatattttatccacacacacaccttaaaatttttagattaaatggcatcaactcttagcttcacacacacacacacaccgaagctggttgttttatttgatttgatataattggtttctctggatggtactttttctttcttggttatttcctattttaaaatatgaaacgcacacaagtcataattattctaatagagcacaattcacaacacgcacatttcaactttaatatttttttagaaacactttatttagtctaattcttaatttttaatatatataatgcacacacacgtttaaatgggcccgcggcccgtttaaacggccggcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacatagcaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatgaaggttaccaaccaaaaggaattgaagcaaaagttgaacgaattgagagaagctcaaaagaagttcgctacctacactcaagaacaagttgacaagatcttcaagcaatgtgccattgctgctgccaaggaacgtatcaacttggccaagttggctgtcgaagaaaccggtattggtttggttgaagacaagatcatcaagaaccacttcgctgctgaatacatctacaacaagtacaagaacgaaaagacctgtggtatcatcgaccacgatgactctttgggtatcaccaaggttgctgaaccaatcggtattgtcgccgccattgtcccaaccactaacccaacttccactgccatcttcaaatctttgatctccttgaagaccagaaacgctatcttcttctccccacacccaagagccaagaagtccaccattgctgctgccaaattaatcttggatgctgctgttaaggctggtgccccaaagaacattattggttggatcgatgaaccttccattgaattgtctcaagacttgatgtctgaagctgatatcatcttggctaccggtggtccatccatggtcaaggccgcttactcttctggtaagccagctattggtgttggtgctggtaacactccagctatcatcgatgaatctgctgacattgacatggctgtctcctccattatcttgtccaagacttatgacaacggtgtcatctgtgcctctgaacaatccatcttggttatgaactctatctacgaaaaggtcaaggaagaatttgttaagagaggttcctacatcttaaaccaaaatgaaattgccaagatcaaggaaaccatgttcaagaacggtgccatcaacgctgacattgtcggtaaatctgcttacatcattgccaagatggctggtattgaagttccacaaaccactaagattttgatcggtgaagttcaatctgtcgaaaagtctgaattattctctcacgaaaagttgtctccagtcttggctatgtacaaggtcaaggatttcgacgaagctttgaagaaggctcaaagattaattgaattaggtggttctggtcacacctcttctctatacattgactctcaaaacaacaaggacaaggtcaaggaattcggtctagctatgaagacttccagaactttcatcaacatgccatcttctcaaggtgcttctggtgatttgtacaactttgccattgctccatctttcactttaggttgtggtacctggggtggtaactctgtttctcaaaacgttgaaccaaagcatttgctaaacatcaagtccgttgctgaaagaagagaaaacatgttgtggttcaaggttccacaaaagatctacttcaaatacggttgtttgagatttgctttgaaggaattgaaagatatgaacaagaagcgtgctttcatcgttactgacaaggatttgttcaaattgggttacgttaacaagatcactaaggttttggatgaaattgatatcaagtactccatcttcactgatatcaaatctgacccaaccattgactccgtcaagaagggtgctaaggaaatgttgaacttcgaaccagataccattatctccattggtggtggttctccaatggatgctgccaaggttatgcatttgttgtacgaatacccagaagctgaaatcgaaaacttggccatcaacttcatggacatcagaaagagaatctgtaacttcccaaagttgggtaccaaggccatttctgttgccattccaaccaccgctggtaccggttctgaagctactccatttgctgtcatcaccaacgacgaaaccggtatgaagtacccattgacctcttacgaattgactccaaacatggccatcattgacactgaattgatgttgaacatgccaagaaagttgactgctgctaccggtattgacgctttagtccacgctatcgaagcttacgtctccgttatggccactgactacactgacgaattggctttgagagctatcaagatgatcttcaagtacttgccaagagcttacaagaacggtactaacgatatcgaagctcgtgaaaagatggctcacgcttccaacattgctggtatggctttcgctaacgctttcttgggtgtttgtcactccatggcccacaagttgggtgctatgcaccacgttcctcacggtattgcttgtgctgttttgattgaagaagtcatcaagtacaacgctactgactgtccaaccaagcaaactgctttcccacaatacaagtctccaaacgccaagagaaagtacgctgaaattgctgaatacttgaacttgaaaggtacttctgacactgaaaaggtcactgctttaatcgaagctatctccaagttgaagattgacttatctattcctcaaaacatctctgctgctggtattaacaagaaggacttctacaacactttagacaagatgtccgaattggctttcgatgaccaatgtaccaccgctaacccaagatacccattgatctctgaattgaaggatatctacatcaagtccttttaagcccgggcgcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccagcattgaaatgatcgaagttcaatgatgaaacggtaattcttctgtcatttactcatctcatctcatcaagttatataattctatacggatgtaatttttcacttttcgtcttgacgtccaccctataatttcaattattgaaccctcacaaatgatgcactgcaatgtacacaccctcatatagtttaaacatttaaatgggccgctctagaggatccccgggtaccgagctcgggcccagcgctactagttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagagacccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtcgagaatttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatcaaagtaaaaaaaagattttcgctacatgttccttttgaagaaagaaaatcatggaacattagatttacaaaaatttaaccaccgctgattaacgattagaccgttaagcgcacaacaggttattagtacagagaaagcattctgtggtgttgccccggactttcttttgcgacataggtaaatcgaataccatcatactatcttttccaatgactccctaaagaaagactcttcttcgatgttgtatacgttggagcatagggcaagaattgtggcttgagatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 15 - DNA, 11114 bp, Artificial sequence
  pBOL120 integration vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccatttaaacggccggccctagatcagagggtggtaaatgaagtgtaatagtattcatttttcttataaatcatcccttccgtgatttatacaaaagaagaggagaatatgctgaatacttggtatattactctacattatactcttatcttgacgggtattctgagcatcttactcagtttcaagatcttttaatgtccaaaaacatttgagccgatctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaaatgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaaagcccgggcggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgtttaaatgtttaaacatggacagatatgcgatgaaaacgctaagtgatactccaaatggtgaaaggtacgatgcttggaaacaatacttggaaatcaccggaaacaccatatgcggcgaaaagccaattagtgtgatactaagtgctttatcgaaaatccgtgatgccggtccttcaggcatcaaatttcagtggcctaattattcacagagttctcatgtgacaagtattgatgatagtagtgtcagttatgcttcaggttatgttactataggataatgatcacggctaaaacggtcgaatgtaagcatatatctttcgattgtataattgttcccaaatactacagcatctcaaggaaaaaaaaacaaaaacttccaaaaaaatcgaatccctgaggaatctttaatacattttcaatctatttaagttttataaacgtgtatatgagatgtcatgagcatgaattattaataataaaaactaaatcattaaagtaacttaaggagttaaagcccgggctttaattgttagcagccttgacttgagcaatcaattctggaacaaccttgttgacatcaccgacaatggccaaatcagcaaccttcatgattggagcttcgacatctttgttgatggcaatgatgtagtcagagtcttgcataccagccaagtgttggatggcaccagagataccacaagcaatgtacaaagttggtctgacggtcttaccggtttgaccgacttgcaagtccttgtcaacccattccttttcaatggcagctctggaagcagcaatggtaccacccaacaaagaagctaattcttccaatttttcgaagttttccttggaaccaacaccacgaccaccagcaaccaaaaccttggcttcaccgatatcagcaatgtccttggccaatttgacaaccttggaaaccttggttctgatatcagaagcagtcaatttgatggcaaccttttcgatcttgtcatcagaaacgttagcatcgttaactggcaatttttcaaagacacctggtctgacggtggccatttgaggtctgtggtcagaacagacaatggtagcaatcaagttaccaccgaaagctggtctggtagccaacaagtcacggttttcgacatcgatatccaaagaggtacagtcagcagtcaaaccagtagacaatctggcagcaattcttggacccaagtctctaccgatgaaagtagcaccgatgaataagatttctggctttctttcgttgaccaagtcacagataaccttggcgtaaccgtcagtggagaaatgagctaataattcgttgtcagcagccaaaaccttgtcagcaccgtgggacaacaagtccttggacatcttttcagtgttgtgacccaataagacagcagtcaattcaacacccaatttttcagccatttccttacccttacctagcaattccaaagaaaccttttgtaattcaccatctctttgttcagcgaaaacccagacacccttgtagtcagccttgttcatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaggccggccgtttaaacgggccgccaccgcggtggagcctgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgctcgagtcgactgcagtttatctaatggagaaaccatcagtcaagacacatcttcttcttctagcgaagtgacgggcagtggtagtaccttcaccagttggagtagcaatggtgaaagtggtggaaccttcacctctgaaacctaaaccagcgaaagatggaccgttcttgacaaagatggaggtttgcatgtcacgggcagccttgttcaatctggagatgttttgagagtgcatggtagcagtgtgatgtagaccttgttccaattcaatggcaacttccaaagcttcatcgaagtctggaactctgacaactggaacaattggcatcaacaattcaacagtagcgaatgggtgggacttttcagtttcgacaatgatcaatcttggggtgaaatcacaagcaataccagcttctttcaagatttcagtggcagacttaccaaccaatttcttgttggtgacacccttgtcagtgacggcaaccttttccaatttttggatatcagatgggttagtgacgtgcaaagcaccgttcttttccatttggaacaacaagaagtcagcaatggagtcaacggcaacaacagacttttcagcgatacacaagatattatggtcaaaggaagcaccgtcgacaatgtcagcagcagccttttcaatgttagcggtttcgtcaacgatggatggagggttaccagcaccagcaccgataaccttcttaccagattgcatagcttgcaagacaacacctggaccaccagtgatgaccaacaatggaacctttgggtggttcatcatttcttgagcagcttggatagatggcttggcaacggtgacaatcaagttgtcaataccacaagaatctctgacgatagtgttcaacttttcaatcaaccataaagagatgttcttggcacctgggtgaggagagtagaaaacggcgttaccagcagccaacataccgatggagttacagatcaaagtttcagttgggttggtagatggagcaacagcaccgatgacaccgtatggagataattcgtataaagtcataccgttgtcaccggtagcaacttcagtgtacaagtcttcaacacctggagtcttttcgatagctaaagtgttcttcaagattttatcggtgacattacccataccggtttcagcaacagctctggtagcaatggtttcgatttctgggtataaagcttctctgatggccttgacaacgtttcttctttcttccaaagatttttccttgtaacagttttgagcaatgacggcagcttggacagcttcatcgacggtatcgaaaacaccggacttggcaccttgggtggtggtcttggttggaacttccttttgttcagccaatttttccaacaaaaccttcttgactaattgttccaattccaaagattccattttttactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 24 bp, Artificial sequence
  P1 primer
gaattgaaggatatctacatcaag

SEQ ID NO: 17 - DNA, 25 bp, Artificial sequence
  P2 primer
cccatctacggaaccctgatcaagc

SEQ ID NO: 18 - DNA, 26 bp, Artificial sequence
  P3 primer
gatggtgtcaccattaccaggtctag

SEQ ID NO: 19 - DNA, 56 bp, Artificial sequence
  P4 primer
gttctctggtcaagttgaagtccattttgattgatttgactgtgttattttgcgtg

SEQ ID NO: 20 - DNA, 25 bp, Artificial sequence
  P5 primer
gaacaatagagcgaccatgaccttg

SEQ ID NO: 21 - DNA, 25 bp, Artificial sequence
  P6 primer
gacatcagcgtcaccagccttgatg

SEQ ID NO: 22 - DNA, 27 bp, Artificial sequence
  P7 primer
gattgaaggtttcaagaacaggtgatg

SEQ ID NO: 23 - DNA, 25 bp, Artificial sequence
  P8 primer
ggcgatcagagttgaaaaaaaaatg

(19) United States
(12) Patent Application Publication
(10) Pub. No.: US 2010/0205857 A1
(43) Pub. Date: Aug. 19, 2010

(54) BUTANOL PRODUCTION IN A EUKARYOTIC CELL

(76) Inventors: Einte-Karst Dijk, Rotterdam (NL); Lourina Madeleine Raamsdonk, Nootdorp (NL)

Correspondence Address: NIXON & VANDERHYE, PC, 901 NORTH GLEBE ROAD, 11TH FLOOR, ARLINGTON, VA, 22203, US

(21) Appl. No.: 12/669,251

(22) PCT Filed: Jul. 11, 2008
(86) PCT No.: PCT/EP2008/059120

§ 371 Date: Jan. 15, 2010

Related U.S. Application Data
(60) Provisional application No. 60/935,031, filed on Jul. 23, 2007.
(60) Provisional application No. 61/064,120, filed on Feb. 19, 2008.
(60) Provisional application No. 61/064,118, filed on Feb. 19, 2008.
(60) Provisional application No. 61/048,700, filed on Apr. 29, 2008.

(30) Foreign Application Priority Data
Jul. 23, 2007 (EP) 07112956.3
Dec. 21, 2007 (EP) 07123976.8
Feb. 19, 2008 (EP) 08101747.7
Feb. 19, 2008 (EP) 08101748.5
Apr. 29, 2008 (EP) 08155396.8

(51) Int. Cl. C12P 7/16; C12N 1/16; C12N 1/19; C12N 15/81; C07C 29/74; C10L 1/182

(52) U.S. Cl. 44/452; 435/255.1; 435/254.2; 435/255.2; 435/471; 435/160; 568/913

(57) ABSTRACT

The present invention relates to a eukaryotic cell capable of producing butanol and ethanol at a ratio butanol:ethanol of between 1:2 to 1:100. The present invention further relates to a process for the preparation of butanol and ethanol comprising fermenting a eukaryotic cell in a suitable fermentation broth, wherein butanol and ethanol are produced at a ratio butanol:hethanol of between 1:2 to 1:100 and a process for the recovery of butanol and ethanol from an aqueous solution comprising butanol and ethanol wherein the ratio butanol:ethanol is between 1:2 to 1:100 comprising, separating an ethanol/butanol/water mixture from the aqueous solution; separating an ethanol/water mixture from the ethanol/butanol/water mixture; separating a butanol/water mixture from the ethanol/butanol/water mixture; and recovering of butanol and ethanol.

### FIELD OF THE INVENTION

The present invention relates to a eukaryotic cell capable of producing butanol and ethanol, a process for the preparation of butanol and ethanol and a process for the recovery of ethanol and butanol from an aqueous solution.

Ethanol is currently the largest alternative (bio)fuel which is predominantly made by large-scale yeast fermentation of sugars followed by separation of the ethanol by distillation. The main sugars used in ethanol fermentation are predominantly derived from sugar cane or maize. Since the prices for ethanol may fluctuate considerably, there is a need for producing an alternative product in the same ethanol production facilities.

An attractive product that may be produced in an ethanol fermentation process is butanol. Butanol is suitable as an alternative engine fuel. Butanol has a higher energy content than ethanol, is less corrosive than ethanol and can be transported through existing pipelines and filling stations of fossil fuels. Butanol also finds use as an important industrial chemical such as a solvent for a wide variety of chemical and textile processes, in the organic synthesis of plastics, as a chemical intermediate and as a solvent in the coating and food and flavor industry.

Biological synthesis of butanol and ethanol can be achieved by fermentation using the acetone-butanol-ethanol (ABE) process carried out by the bacteria Clostridium acetobutylicum or other Clostridium species, wherein the ratio A:B:E is generally 3:6:1. However, Clostridium fermentations are not attractive to be carried out on a large scale, because they require sterile process conditions and generally are susceptible to bacteriophage infection. Another disadvantage is that Clostridium fermentations need to be performed under strict anaerobic conditions.

Eukaryotic cells, such as yeast, provide a very suitable alternative, because eukaryotic cells are not susceptible to phage infection or other infection since eukaryotic based fermentation processes can be run at low pH. Therefore, the use of a eukaryotic cell does not require a sterile process, thereby lowering the cost price of a product of interest.

A butanol producing yeast is known from WO2007/041269. WO2007/041269 discloses a recombinant Saccharomyces cerevisiae, which is transformed with at least one DNA molecule encoding a polypeptide that catalyses one of the reactions of the butanol pathway. However, the amount of butanol produced by this genetically modified Saccharomyces strain known in the art is still insufficient for an economically attractive process for the production of butanol and ethanol.

The aim of the present invention is an improved process for the production of butanol and ethanol in a eukaryotic cell.

The aim is achieved according to the present invention with a eukaryotic cell capable of producing butanol and ethanol at a ratio butanol:ethanol of between 1:2 to 1:100.

Surprisingly, it was found that a eukaryotic cell according to the present invention may advantageously be used in an ethanol fermentation process, for instance a large-scale ethanol production process, with minor to no adaptation in fermentation and distillation equipment and resulting in only a slightly lower yield of solvent (butanol plus ethanol) (g solvent/g sugar) in comparison with the yield of solvent in an ethanol fermentation process, wherein the solvent is only ethanol. In addition, since butanol is more toxic to a eukaryotic cell than ethanol, it was found advantageous that ethanol is produced in addition to butanol to result in an economic yield of solvent (butanol+ethanol), as compared to a fermentation process, wherein butanol is the only solvent.

The ratio butanol:ethanol produced by the eukaryotic cell according to the present invention preferably is between 1:3 and 1:80, preferably between 1:3 and 1:50, 1:4 to 1:40, 1:5 to 1:30, preferably between 1:5 and 1:20, or between 1:5 to 1:15 or between 1:6 to 1:14, or between 1:7 to 1:13, or more preferably between 1:8 to 1:12, most preferably between 1:9 to 1:11. As used herein, the ratio butanol:ethanol is based on a w/w ratio.

Surprisingly, it was found that when a eukaryotic cell according to the present invention produces butanol and ethanol at a preferred ratio, the natural poor solubility of butanol in water can be used for separating butanol from the fermentation broth.

Preferably, the amount of butanol produced by the eukaryotic cell in the fermentation broth according to the present invention is at least 0.4 g/l, preferably 0.5 g/l, preferably at least 0.6, 0.7, 0.8, 0.9, 1, 2, 3, 4, 5, 6, 7, 8, 9, 10 or at least 15 or 20 g/l butanol, and usually below 30 g/l.

As used herein the wording butanol is used to indicate n-butanol or 1-butanol.

A eukaryotic cell according to the present invention commonly is a recombinant eukaryotic cell. A recombinant eukaryotic cell is defined as a cell which contains a nucleotide sequence and/or protein, or is transformed or genetically modified with a nucleotide sequence that does not naturally occur in the yeast, or it contains additional copy or copies of an endogenous nucleic acid sequence (or protein), or it contains a mutation, deletion or disruption of an endogenous nucleic acid sequence.

Preferably, the eukaryotic cell according to the present invention has a high tolerance towards butanol and ethanol.

The eukaryotic cell capable of producing butanol according to the present invention may be any suitable eukaryotic cell comprising any suitable pathway for producing butanol. A suitable pathway may for instance be a non-fermentative pathway for 2-keto acid degradation to alcohols as disclosed by Atsumi et al. (2008), Nature Letters, Vol. 451, p. 86-90. Preferably, a eukaryotic cell capable of producing butanol comprises one or more enzymes that produce acetoacetyl-CoA, 3-hydroxybutyryl-CoA, crotonyl-CoA, butyryl-CoA, butyrylaldehyde and butanol.

Suitable enzymes that catalyse the formation of these products are for instance acetyl-CoA acetyltransferase or thiolase (E.C. 2.3.1.9) (SEQ ID NO:1), 3-hydroxybutyryl-CoA dehydrogenase (E.C. 1.1.1.1.57) (SEQ ID NO:2), 3-hydroxybutyryl-CoA dehydratase (E.C. 4.2.1.55) (SEQ ID NO:3), butyryl-CoA dehydrogenase (E.C. 1.3.99.2) (SEQ ID NO:4), NAD(P)H-dependent butanol dehydrogenase (E.C. 1.1.1.-) (SEQ ID NO:5) and alcohol/aldehyde dehydrogenase (E.C. 1.1.1.1/E.C. 1.2.1.10) (SEQ ID NO:6), The enzymes of the butanol pathway may be homologous and/or heterologous to the eukaryotic cell. The enzymes may for instance be derived from a Clostridium sp. for instance Clostridium acetobutylicum or Clostridium beijerinckii.

A eukaryotic cell according to the present invention may be any suitable microbial cell, preferably a yeast or filamentous fungus. Preferably, a eukaryotic cell belongs to a genus of Pichia, Kluyveromyces, Candida, Saccharomyces, Yarrowia, or Rhizopus. A more preferred eukaryotic cell belongs to a species Pichia stipidis, Kluyveromyces lactis, Yarrowia lipolytica, Brettanomyces bruxellensis, Zygosaccharomyces bailii. Preferably, a eukaryotic cell according to the present invention is a yeast cell, preferably a Saccharomyces sp., preferably a Saccharomyces cerevisiae.

Preferably, a eukaryotic cell according to the present invention is a eukaryotic cell comprising at least one inactivated nucleotide sequence encoding an enzyme that is required for the production of ethanol. Preferably, the at least one inactivated nucleotide sequence encodes an alcohol dehydrogenase.

Inactivation of a nucleotide sequence encoding an enzyme, may be achieved by mutation, deletion or disruption of (part of) a nucleotide sequence.

In another embodiment, a eukaryotic cell according to the present invention preferably comprises a nucleotide sequence encoding a butyryl-CoA dehydrogenase and at least one nucleotide sequence encoding a heterologous electron transfer flavoprotein (ETF). A heterologous electron transfer flavoprotein in the eukaryotic cell according to the present invention may be derived from any suitable origin. Preferably, the ETF is derived from the same origin as the butyryl-CoA dehydrogenase. Preferably, the ETF is derived from prokaryotic origin preferably from a Clostridium sp., preferably a Clostridium acetobutylicum wherein the ETF comprise two subunits: an alpha (SEQ ID NO:7) and a beta subunit (SEQ ID NO:9).

Preferably, a eukaryotic cell according to the present invention further comprises a nucleotide sequence encoding a heterologous enzyme having enzymatic activity for converting pyruvate, acetaldehyde or acetate into acetyl-CoA in the cytosol.

It may be preferred that a heterologous enzyme having enzymatic activity for converting pyruvate, acetaldehyde or acetate into acetyl-CoA in the cytosol is an enzyme which catalyses the conversion of pyruvate to acetyl-CoA, such as a pyruvate:NADP oxidoreductase (E.C. 1.2.1.51).

Alternatively, a eukaryotic cell comprises a nucleotide sequence encoding a heterologous enzyme that catalyses the conversion from acetate to acetyl-CoA such as an acetyl-CoA synthetase (E.C. 6.2.1.1)

Preferably, a eukaryotic cell according to the present invention comprises a nucleotide sequence encoding a heterologous enzyme that catalyses the conversion of acetaldehyde into acetyl-CoA, preferably an acetylating acetaldehyde dehydrogenase (E.C.1.2.1.3, E.C. 1.2.1.4 or E.C. 1.2.1.5).

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but have been obtained from another cell or synthetically or recombinantly produced.

The term “nucleotide sequence” as used herein, includes reference to a deoxyribonucleotide or ribonucleotide polymer, i.e. a polynucleotide, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skilled in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

In another aspect the present invention relates to a process for increasing the butanol production in a eukaryotic cell capable of producing butanol according to the present invention comprising subjecting a population of eukaryotic cells capable of producing butanol to mutagenesis; and selecting a population of mutant eukaryotic cells for increased butanol production. Preferably, the mutagenesis is carried out such that 20% of a population of mutant eukaryotic cells shows an increased butanol production as compared to a starting population of eukaryotic cells.

Mutagenesis may be carried out by various methods known in the art, for instance ultraviolet light (UV) mutagenesis, ionizing radiation or the use of mutagentia. Suitable mutagentia are ethyl methanesulfonate (EMS), diethyl sulfate (DES), methyl methanesulfonate (MMS), dimethyl sulfate (DMS), nitroquinoline oxide (NQO), nitrosoguanidine (NTG), nitrogen mustard (HN2), β-propiolactone, nitrous acid, nitrosoimidazolidone (NIL) and tritiated uridine. Preferably, the mutagenesis comprises incubating a population of eukaryotic cells in the presence of NTG between 30 and 60 min, preferably between 40 and 50 min; and selecting a population of mutant eukaryotic cells for increased butanol production. Preferably said incubation of eukaryotic cells in the presence of NTG and selecting mutant eukaryotic cells for increased butanol production is repeated one, two or three times.

Preferably, a eukaryotic cell according to the present invention comprises a mutation resulting in an increased butanol production as compared to a cell which does not comprise said mutation, which may be obtainable by the process for increasing the butanol production according to the present invention.

Surprisingly, the amount of butanol produced by the eukaryotic cell that was subjected to a process for increasing the butanol production according to the present invention was increased with at least 5% preferably at least 10%, preferably at least 20 or 40% compared to a eukaryotic cell that was not subjected to said mutagenesis.

Preferably, a eukaryotic cell according to the present invention is a Saccharomyces cerevisiae with deposit number CBS 122885, or a Saccharomyces cerevisiae with deposit number CBS 123039.

Preferably, the eukaryotic cell according to the present invention is able to grow on any suitable carbon source and/or ferment it into butanol and ethanol. Suitable carbon sources are, celluloses, hemicelluloses, pectines, rhamnose, glucose, galactose, fucose, xylose, arabinose, maltose, maltodextrines, ribose, ribulose, or starch, starch derivatives, sucrose, lactose and glycerol.

In another aspect the present invention relates to a process for the preparation of butanol and ethanol comprising fermenting a eukaryotic cell according to the present invention in a suitable fermentation broth, wherein butanol and ethanol are produced at a ratio butanol:ethanol of between 1:2 to 1:100. Preferred ratios of butanol:ethanol in a process for the preparation of butanol and ethanol according to the present invention and a preferred amount of butanol produced are as described herein above,

Surprisingly, it was found that the process according to the present invention could advantageously be applied in a large scale ethanol fermentation plant with minor to no adaptions in fermentation equipment and no additional energy requirement in the recovery of ethanol and butanol.

Although it is known that a eukaryotic cell such as Saccharomyces cerevisiae produces ethanol and butanol, we are the first who understood that fermenting a eukaryotic cell capable of producing butanol and ethanol on an industrial scale is an economically attractive process. Therefore, the process for the production of butanol and ethanol according to the present invention is preferably, carried out on an industrial scale.

Industrial scale is used herein to indicate a process for the preparation of butanol and ethanol that is carried out in a (fermentation) volume of at least 1, 2, 4, 5, 10 m³ (cubic metre), preferably at least 20, 30, 50 or at least 100 m³ (cubic metre), 200 or 500 m³ or at least 1000 to 2000 m³. The eukaryotic cell in the process for the preparation of butanol and ethanol may be any suitable eukaryotic cell capable of producing butanol according to the present invention as described herein above.

The fermentation broth in the process for the preparation of butanol and ethanol according to the present invention may comprise any suitable nutrient necessary for growth of a specific eukaryotic cell and for the fermentative production of butanol and ethanol. The essential nutrients for growth of a eukaryotic cell and for production of buatbnol are known to the skilled man in the art. Preferably, the fermentation broth comprises a carbon source such as celluloses, hemicelluloses, pectines, rhamnose, glucose, galactose, xylose, arabinose, fucose, fructose, maltose, maltodextrines, ribose, ribulose, or starch, starch derivatives, sucrose, lactose, fatty acids, triglycerides and glycerol. The carbon source may be derived from sugar beet, sugar cane or maize, but is not limited thereto, preferably a cellulose or hemicellulose containing source. Preferably, the fermentation broth comprises a nitrogen source such as ureum, or an ammonium salt such as ammonium sulphate, ammonium chloride, ammonium nitrate or ammonium phosphate.

The fermentation process for the production of butanol and ethanol according to the present invention may be an aerobic or an anaerobic fermentation process.

An anaerobic fermentation process is herein defined as a fermentation process run in the absence of oxygen or in which substantially no oxygen is consumed, preferably less than 5, 2.5 or 1 mmol/L/h, and wherein organic molecules serve as both electron donor and electron acceptors. The fermentation process according to the present invention may also first be run under aerobic conditions and subsequently under anaerobic conditions.

The fermentation process may also be run under oxygen-limited, or micro-aerobical, conditions. Alternatively, the fermentation process may first be run under aerobic conditions and subsequently under oxygen-limited conditions. An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used. Preferably, in a process under oxygen-limited conditions, the rate of oxygen consumption is about 5.5, more preferably about 6 and even more preferably about 7 mmol/L/h.

The process for the production of butanol and ethanol according to the present invention may be run at any suitable temperature, preferably between 10 and 45 degrees Celsius, preferably 15 and 40, preferably between 20 and 35, or between 25 and 40 degrees Celsius. The process for the production of butanol and ethanol according to the present invention may be carried out at any suitable pH value, for instance between 2 and 9, preferably between 2.5 and 8. The pH in the fermentation broth preferably has a value of below 7.6, preferably below 5.5, preferably below 5, preferably below 4.5, preferably below 4, preferably below pH 3.5 or below pH 3.0, preferably above pH 2.5.

In a preferred embodiment, the process for the production of butanol and ethanol according to the present invention comprises separating butanol from ethanol to obtain ethanol containing less than 1% w/w preferably less than 0.5% w/w of butanol, and butanol containing less than 1% w/w, preferably less than 0.5% w/w of ethanol. Preferably, said separating of butanol from ethanol is carried out by distillation as described herein below.

In a preferred embodiment of the process according to the present invention, butanol and ethanol are recovered from the fermentation broth.

Recovery of butanol and ethanol may be carried out by any suitable method known in the art, for instance distillation, adsorption, vacuum extraction, solvent extraction, or pervaporation. Preferably, butanol and ethanol are recovered from the fermentation broth by distillation. Preferably, butanol and ethanol produced in the process for the preparation of butanol and ethanol according to the present invention are recovered by a process for the recovery of butanol and ethanol as described herein below.

In another aspect the present invention relates to a process for the recovery of butanol and ethanol from an aqueous solution comprising butanol and ethanol at a ratio butanol:ethanol of between 1:2 to 1:100 comprising separating of an ethanol/butanol/water mixture from the aqueous solution; separating an ethanol/water mixture from the ethanol/butanol/water mixture; separating a butanol/water mixture from the ethanol/butanol/water mixture; and recovering of butanol and ethanol.

Surprisingly it was found that the process for the recovery of butanol and ethanol from an aqueous solution comprising butanol and ethanol according to the present invention may advantageously be used in a large-scale ethanol fermentation process with minor adaptations in distillation equipment. The aqueous solution comprising butanol and ethanol may be any suitable aqueous solution. Preferably, the aqueous solution is a fermentation broth. The fermentation broth may be any a fermentation broth comprising butanol and ethanol at a ratio of 1:2 to 1:100. Preferably the fermentation broth is obtained by a process for the preparation of butanol and ethanol comprising fermenting a eukaryotic cell according to the present invention as disclosed herein above. The aqueous solution preferably comprises butanol and ethanol at preferred ratios of butanol: ethanol as defined herein above.

Separating an ethanol/butanol/water mixture from the aqueous solution comprising butanol:ethanol at a ratio of 1:2 to 1:100 is usually carried out by distillation (stripping), usually in a column, at any suitable temperature, which may depend on the concentration and ratio of butanol and ethanol in the aqueous solution. Preferably separating of an ethanol/butanol/water mixture by distillation is carried out in a column wherein the bottom temperature is between 90 to 110, preferably between 95 and 105, preferably between 98 and 102, preferably between 99 and 101 degrees Celsius. Preferably, the temperature at the top of a column for separating (distilling) an ethanol/butanol/water mixture in the process for the recovery of the invention is carried out between 70 and 90, preferably between 75 and 85, preferably between 78 and 83, preferably between 79 and 81 degrees Celsius. The top and bottom temperature of a column for distilling a butanol/ethanol/water mixture usually depends on the concentration and ratio of butanol and ethanol in the ethanol/butanol/water mixture and operating pressure.

The ethanol/butanol/water mixture that is separated from the aqueous solution may comprise any suitable concentration of solvent. As used herein, solvent is defined herein as the sum of ethanol and butanol. Preferably, the ethanol/butanol/water mixture comprises between 40 and 80 wt % of solvent, preferably between 45 and 75 wt %, preferably between 50 and 70 wt %, preferably between 55 and 65 wt %, preferably between 58 and 63 wt % of solvent.

Separating an ethanol/water mixture from an ethanol/butanol/water mixture is usually carried by distillation, usually in a column, wherein the bottom temperature is between 90 to 110, preferably between 95 and 105, preferably between 98 and 102, preferably between 99 and 101 degrees Celsius. The temperature at the top of a column for separating an ethanol/water mixture by distillation is between 70 and 90, preferably between 75 and 85, preferably between 78 and 83, preferably between 79 and 81 degrees Celsius. The top and bottom temperature of a column for separating an ethanol/water mixture usually depends on the concentration and ratio of butanol and ethanol in the ethanol/butanol/water mixture and the operating pressure.

Separating of a butanol/water mixture from an ethanol/butanol/water mixture is usually carried out by distillation, usually in a column. Preferably, the bottom temperature of a column for separating a butanol/water mixture is between 100 and 140 degrees Celsius, for instance between 105 and 135, 110 and 130, 115 and 128, between 118 and 127, or between 119 and 125 degrees Celsius. The temperature at the top of a column for separating a butanol/water mixture by distillation preferably has a temperature of between 70 and 100 degrees Celsius, for instance between 75 and 95, between 78 and 90 degrees Celsius. The top and bottom temperature of a column for separating a butanol/water mixture usually depends on the concentration and ratio of butanol and ethanol in the ethanol/butanol/water mixture and the operating pressure.

In the event separation of a butanol/water mixture is carried out after separation of an ethanol/water mixture, the separation of a butanol/water mixture is carried out from a (second) ethanol/butanol/water/mixture which usually comprises an increased ratio of butanol as compared to ethanol.

The separation of a butanol/water mixture, preferably by distillation, from an ethanol/butanol/water mixture may comprise bringing part of the ethanol/butanol/water mixture to a temperature of between 10 and 40, preferably between 20 and 30 degrees Celsius. Preferably, the part of the ethanol/butanol/water mixture that is brought to said temperature range comprises a higher amount of butanol than ethanol, preferably between 5 and 90, 10 and 80, 20 and 70, preferably between 30 and 60, preferably between 40 and 60 wt % butanol, and between 0.1 and 10, such as between 1 and 5 wt % of ethanol. Preferably, the cooled ethanol/butanol/water mixture, i.e. an ethanol/butanol/water mixture that is brought to a temperature range of between 10 and 40 degrees Celsius is fed to an immiscible liquid liquid separator, preferably a gravitational decanter. It was found advantageous to bring part of an ethanol/butanol/water mixture to a temperature of between 10 and 40, since this resulted in an efficient recovery of butanol with minor investment and adaptation of an ethanol recovery process.

The ethanol and butanol in the process for the recovery of ethanol and butanol according to the present invention may be recovered in any suitable form. The ethanol and/or butanol may comprise water, a so-called hydrous ethanol and/or hydrous butanol. Hydrous ethanol or hydrous butanol typically comprises at least 2 v/v % of water for instance at least 5, 10, 20 or 30% v/v of water, but usually below 50% v/v of water.

Alternatively, the ethanol and/or butanol may be recovered in an anhydrous form, i.e. the ethanol and/or butanol comprises 1 v/v % or less water. In the event the butanol and/or ethanol are recovered in an anhydrous form, the process for the recovery of butanol and ethanol according to the present invention further comprises drying of the ethanol water mixture and/or the butanol/water mixture and/or the ethanol/butanol/water mixture to obtain anhydrous ethanol and/or anhydrous butanol. Drying may be carried out by known techniques such as entrainer distillation, molecular sieving, membrance gas separation or pervaporation.

The different distillation steps and/or drying in the process for the recovery of butanol and ethanol according to the present invention may be carried out in any suitable order. Preferably, the process for the recovery of butanol and ethanol according to the present invention comprises as a first step of seperating an ethanol/butanol/water mixture from an aqueous solution. Separation of an ethanol/water mixture and a butanol/water mixture may be carried out concomitantly or successively.

The process for the recovery of butanol and ethanol according to the present invention may be carried out at any suitable pressure, preferably at atmospheric pressure. It is to be understood that the preferred temperature ranges for distillation (separation) of the different mixtures as defined herein above may be adapted when the pressure during distillation deviates from atmospheric pressure. The relationship between pressure and temperature is known to a skilled person in the art.

Preferably, the process for the recovery of butanol and ethanol is carried out at an industrial scale. Preferably, a process for the recovery of butanol and ethanol at an industrial scale comprises a column for distillation that may comprise 5 to 60 theoretical stages, preferably 10 to 50, preferably 15 to 30 theoretical stages. A theoretical stage is a common understanding for a skilled person in the field of distillation technology. Preferably, a column for distillation has a diameter of 0.5 to 10, preferably between 1 and 5 metres. The different distillations may comprise columns of different sizes.

The butanol recovered from the aqueous solution may be purified. Purification of butanol may be carried out by distillation or any other separation step known to those skilled in the art.

The following descriptions of the figures show preferred embodiments of a process for the recovery ethanol and butanol according to the present invention.

FIG. 1 shows a block diagram illustrating the production of ethanol and butanol via fermentation and recovery thereof. Apparatus 1 comprises a eukaryotic cell in an aqueous environment capable of producing ethanol and butanol under conditions known to the skilled man in the art. Stream 046 consists of all the required substrates required for ethanol and butanol production by and growth of a eukaryotic cell. Stream 048 leaving apparatus 1 is an aqueous stream comprising butanol and ethanol at a ratio butanol:ethanol of 1:2 to 1:100. Stream 048 is fed to an apparatus 2 where the solvents are recovered form the water stream. Most of the water leaves apparatus 2 as stream 049, whereas ethanol and butanol leave apparatus 2 as stream 045 and 047, respectively.

FIG. 2 shows a block diagram, which shows a preferred embodiment of apparatus 2 from FIG. 1. Aqueous stream 010 comprising ethanol and butanol is fed into column 3. Stream 010 can be preheated before entering the column by for example exchanging heat with stream 002. In column 3 the solvents are stripped form the stream 010 resulting in an aqueous bottom stream 002 without solvents and a solvent rich vapor stream 001 at the top of column 3. Column 3 comprises sufficient theoretical stages for the separation of ethanol and butanol. Heat can be supplied to column 3 via a reboiler or direct steam injection. Optionally a small condenser is used to create some reflux. In that case stream 001 will be a side stream drawn close from the top of the column. The vapor stream 001 leaving column 3 is fed to column 4, which comprises sufficient stages to concentrate ethanol without butanol at the top of the column via refluxing. An ethanol water mixture near the azeotrope leaves the top of column 4 as stream 003. A butanol rich liquid side stream 006 is drawn from the column and after cooling to 10-40° C. fed to an immiscible liquid liquid separator 5, preferably a gravitational decanter. The cooled liquid will separate in stream 009 with 8-12 wt % solvent and stream 007 with approximately 70-80 wt % solvent. Layer 009 is fed to column 3 and layer 007 is recycled to column 4 at a tray below the side stream extraction tray. Optionally the hot stream 006 is used to heat other cold streams. Dehydrated butanol is drawn from the bottom of column 4 as stream 004. Optionally higher alcohols (fusels) are drawn from column 4 as side stream 005. Heat is supplied to column 4 via a reboiler. In the event the butanol stream 004 comprises impurities, it may be preferred to feed stream 004 to column 6, where via refluxing, components with a higher boiling temperature than butanol are separated from the butanol as bottom stream 008. Butanol is collected as top product of column 6 as stream 050. A reboiler is used to supply heat to column 6.

Another preferred embodiment of apparatus 2 for the recovery of butanol and ethanol from FIG. 1 is shown in FIG. 3. FIG. 3 shows a block diagram wherein an aqueous stream 044 comprising ethanol and butanol is fed into column 7. Stream 044 can be preheated before entering the column by for example exchanging heat with stream 036. In column 7 the solvents are stripped from stream 044 resulting in an aqueous bottom stream 036 without solvents and a solvent rich vapor stream 035 at the top of column 7. Column 7 has sufficient theoretical stages for the separation. Heat can be supplied to column 7 via a reboiler or direct steam injection. Optionally a small condenser is used to create some reflux. In that case stream 035 will be a side stream drawn close from the top of the column. The vapor stream 035 leaving column 7 is fed to column 8 with sufficient theoretical stages to concentrate ethanol without butanol at the top of the column via refluxing. An ethanol water mixture near the azeotrope is drawn from the top of this column as stream 037. A butanol rich liquid side stream 040 is drawn from the column and after cooling to 20 to 30° C. fed to an immiscible liquid liquid separator 9, preferably a gravitational decanter. The cooled liquid will separate in stream 043 with approximately 8 to 12 wt % solvent and stream 041 with approximately 70 to 80 wt % solvent. Stream 043 is recycled to column 8 at a tray above the side stream extraction tray. Stream 041 is fed to column 10 where under reflux conditions butanol is dewatered. The dewatered butanol leaves column 10 as bottom stream 042, optionally this stream is led over a separate column like column 6 to further purify the product. Water and remaining ethanol leaves column 10 as stream 038 and can be recycled to column 7. Optionally the hot stream 040 is used to heat other cold streams. Solvent free water is drawn from the bottom of column 8 as stream 038. Optionally higher alcohols (fusels) are drawn from column 8 as side stream 039. Heat is supplied to column 8 via a reboiler or direct steam injection.

Another preferred embodiment of apparatus 2 for the recovery of butanol and ethanol of FIG. 1 is shown in FIG. 4. FIG. 4 shows a block diagram comprising an aqueous stream 012 comprising ethanol and butanol, which is fed to column 11. Stream 012 can be preheated before entering the column by for example exchanging heat with stream 013. In column 11 the solvents are stripped from stream 012 resulting in an aqueous bottom stream 013 without solvents and a solvent rich vapor stream 011 at the top of column 11. Column 11 comprises sufficient theoretical stages for solvent separation. Heat can be supplied to column 11 via a reboiler or direct steam injection. Optionally a small condenser is used to create some reflux. In this case stream 011 will be a side stream drawn close to the top of the column. The vapor stream 011 leaving column 11 is fed to column 12 with sufficient theoretical stages to concentrate ethanol without butanol at the top of the column via refluxing. An ethanol water mixture near the azeotrope is drawn from the top of this column as stream 014. Optionally higher alcohols (fusels) are drawn from column 4 as side stream 018. Stream 15 leaving the bottom of column 12 contains most of the water and butanol and is fed to column 13. Due to refluxing in column 13, ethanol and some water leave the column as top stream 017. A butanol rich liquid side stream 019 is drawn from the column and is after cooling to 20 to 30° C. fed to an immiscible liquid liquid separator 14, preferably a gravitational decanter. The cooled liquid will separate in stream 023 with approximately 8 to 12 wt % solvent and steam 020 with approximately 70 to 80 wt % solvent. Layer 023 is fed to column 11 and layer 020 is recycled to column 13 at a tray below the side stream extraction tray. Optionally the hot stream 019 is used to heat other cold streams. Dehydrated butanol is drawn from the bottom of column 13 as stream 016. Heat is supplied to column 13 via a reboiler. Stream 016 may be fed to column 15 where via refluxing components with a higher boiling temperature than butanol are separated from the butanol as bottom stream 022. Butanol is collected as top product of column 15 as stream 021. A reboiler is used to supply heat to column 15.

Another preferred embodiment of apparatus 2 in FIG. 1 is shown in FIG. 5. FIG. 5 shows a block diagram comprising an aqueous stream 025 comprising ethanol and butanol which is fed into column 16. Stream 025 can be preheated before entering the column by for example exchanging heat with stream 026. In column 16 the solvents are stripped from stream 025 resulting in an aqueous bottom stream 026 without solvents and a solvent rich vapor stream 024 at the top of column 16. Heat can be supplied to column 16 via a reboiler or direct steam injection. Optionally a small condenser is used to create some reflux. In that case stream 024 will be a side stream drawn close from the top of column 16. The vapor stream 024 leaving column 16 is fed to column 17 with sufficient theoretical stages to concentrate ethanol and butanol at the top of the column via refluxing. An ethanol/butanol/water mixture near the azeotrope is drawn from the top of column 17 as stream 027. Stream 027 is led over a molecular sieves (3 Å) pressure swing system 18 for dehydration. The water rich stream 051 is fed back to column 17 and the dehydrated stream 029 is fed to column 19. Optionally higher alcohols (fusels) are drawn from column 17 as side stream 032. Water leaves column 17 as stream 028. In column 19 stream 029 is via refluxing split into top stream 031 consisting of dehydrated ethanol and bottom stream 030 consisting of dehydrated butanol. A reboiler is used to supply heat to column 19. Stream 030 may be fed to column 20 where via refluxing higher boiling components are separated from the butanol as bottom stream 034. Butanol is collected as top product of column 20 as stream 033. A reboiler is used to supply heat to column 20.

In another aspect the present invention relates to a fermentation broth obtainable by a process for the preparation of butanol and ethanol at a ratio butanol:ethanol of between 1:2 to 1:100 according to the present invention.

The invention also relates to the use of butanol and/or ethanol recovered by a process according to the present invention as a chemical or as a fuel. Examples of the use of butanol as a chemical is the use of butanol as a solvent, for instance in the organic chemistry, or as a raw material for the production of butyl esters or ethers, for instance butyl acrylate. Alternatively, butanol of the inventions may be used as a fuel for instance as an additive to fuels such as gasoline or diesel.

### DESCRIPTION OF THE FIGURES

FIG. 1. Block diagram illustrating a method for producing ethanol and butanol via fermentation in apparatus 1 and separation of the ethanol and butanol from an aqueous stream in apparatus 2.

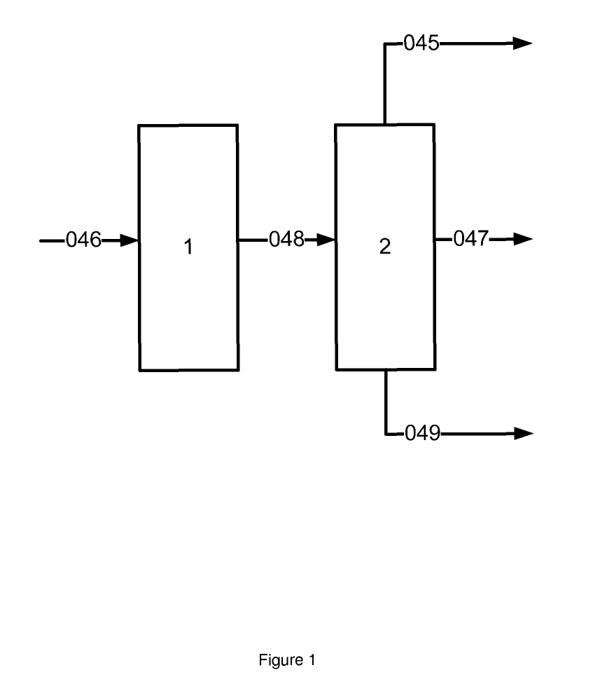

FIG. 2. Block diagram of an example of apparatus 2 of FIG. 1 for the recovery of ethanol and butanol form an aqueous stream wherein ethanol is recovered as hydrous ethanol and hydrous butanol.

FIG. 3. Block diagram of an example of apparatus 2 of FIG. 1 for the recovery of ethanol and butanol form an aqueous stream wherein ethanol is recovered as hydrous ethanol and anhydrous butanol is recovered in anhydrohous form.

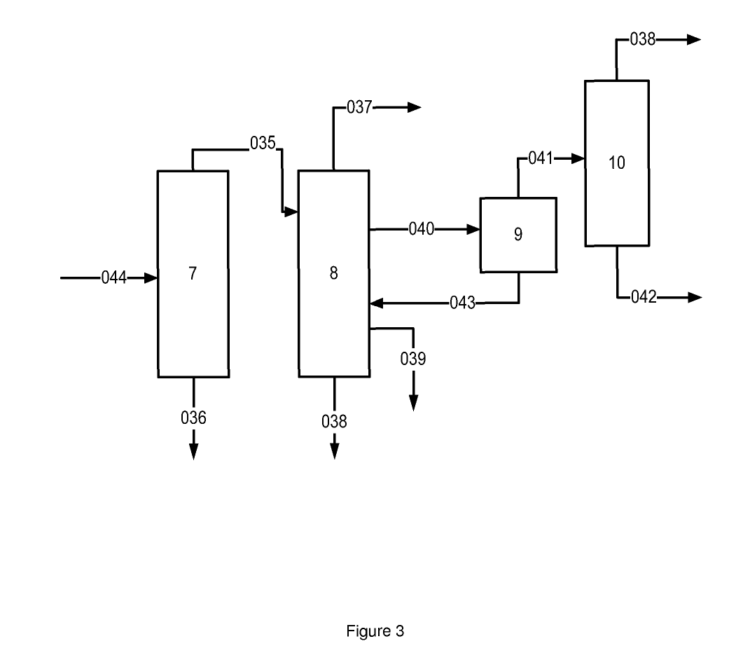

FIG. 4. Block diagram of an example of apparatus 2 of FIG. 1 for the recovery of ethanol and butanol form an aqueous stream wherein ethanol is recovered as hydrous ethanol and hydrous butanol.

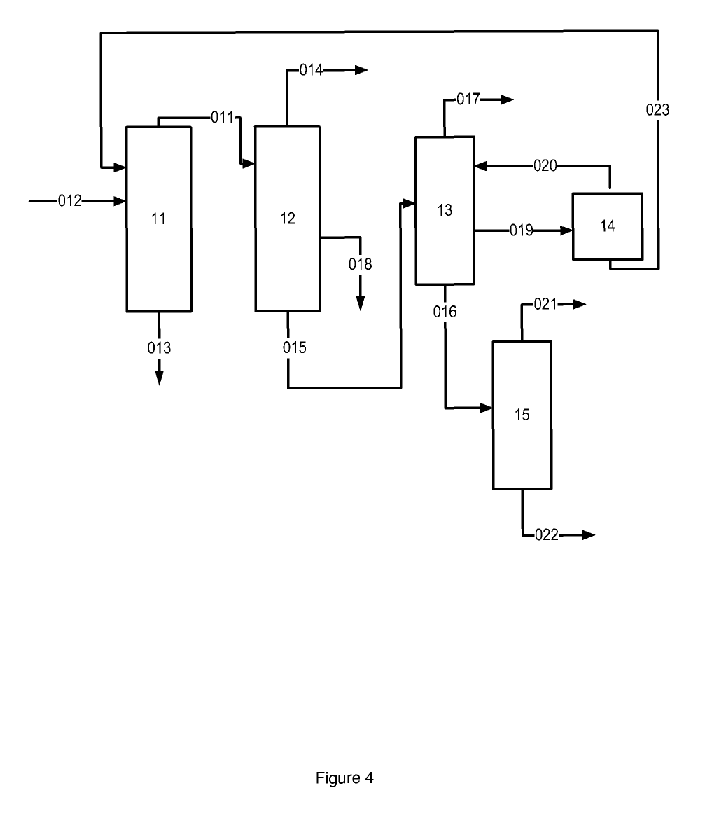

FIG. 5. Block diagram of an example of apparatus 2 of FIG. 1 for the recovery of ethanol and butanol form an aqueous stream wherein anhydrous ethanol is recovered and anhydrous butanol.

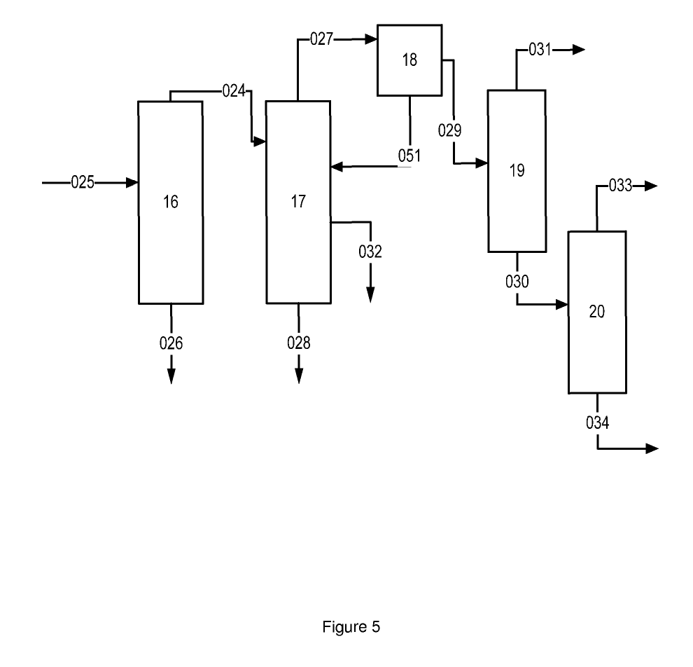

The following examples are for illustrative purposes only and are not to be construed as limiting the invention

### EXAMPLES

### Example 1

### Construction of Saccharomyces cerevisiae Comprising adh Knock Out, ETF and acdh and Subsequent Classical Strain Improvement (CSI)

### 1.1. Construction of a Butanol Producing Yeast Strain and Knocking out the ADH1 and ADH2 Genes

The Clostridium acetobutylicum enzymes involved in butanol biosynthesis from acetyl-CoA used in this experiment are listed in Table 1. The enzymes were codon pair optimized for S. cerevisiae as described in WO2008/000632 and expressed from yeast promoters and terminators as listed in Table 1.

Two yeast integration vectors (pBOL34 [SEQ ID NO:13] and pBOL36 [SEQ ID NO:14]), each containing 3 of the six codon pair optimised genes from Clostridium acetobutylicum involved in butanol biosynthesis, were designed and synthesized at GENEART AG (Regensburg Germany).

The genes ThiL, Hbd and Crt were expressed from pBOL34 containing an AmdS selection marker. The final three genes, Bcd, BdhB and AdhE were expressed from an integration vector with an AmdS selection marker named pBOL36.

TABLE 1
Genes and enzymes used for butanol production in S. cerevisiae
including the promoter (1000 bp) and terminator (500 bp)

| Gene | activity | Promotor | Terminator |
|---|---|---|---|
| ThiL | acetyl CoA c-acetyltransfrase | ADH1 | TDH1 |
| | [E.C. 2.3.1.9] SEQ ID NO: 1 | | |
| Hbd | 3-hydroxybutyryl-CoA | ENO1 | PMA1 |
| | dehydrogenase [E.C.1.1.1.157] | | |
| | SEQ. ID NO: 2 | | |
| Crt | 3-hydroxybutyryl-CoA | TDH1 | ADH1 |
| | dehydratase [E.C.4.2.1.55] | | |
| | SEQ ID NO: 3 | | |
| Bcd | butyryl-CoA dehydrogenase | PDC1 | TDH1 |
| | [E.C.1.3.99.2], | | |
| | SEQ ID NO: 4 | | |
| BdhB | NADH-dependent butanol | ENO1 | PMA1 |
| | dehydrogenase [E.C.1.1.1.—], | | |
| | SEQ ID NO: 5 | | |
| adhE | alcohol/acetaldehyde CoA | TDH1 | ADH2 |
| | dehydrogenase [E.C.1.1.1.1/ | | |
| | E.C.: 1.2.1.10] | | |
| | SEQ ID NO: 6 | | |

For integration in the ADH2 locus, pBOL36 was linearized by a BsaBl digestion. S. cerevisiae CEN.PK113-5D (MATa MAL2-8c SUC2 ura3-52) was transformed with the linear fragment and grown on plates with YCB (Difco) and 5 mM acetamide as nitrogen source.

The AmdS marker was removed by recombination by growing the transformants for 6 hours in YEPD in 2 ml tubes at 30° C. Cells were subsequently plated on 1.8% agar medium containing YCB (Difco) and 40 mM fluoroacetamide and 30 mM phosphate buffer pH 6.8 supporting growth only from cells that have lost the AmdS marker. Correct integration and recombination were confirmed by PCR. The correct integration of the fragment upstream was confirmed with the following primers:

P1:
5′-GAATTGAAGGATATCTACATCAAG-3′
and
P2:
5′-CCCATCTACGGAACCCTGATCAAGC-3′.

The correct integration of the fragment downstream was confirmed with the following primers:

P3:
5′-GATGGTGTCACCATTACCAGGTCTAG-3′
and
P4:
5′-GTTCTCTGGTCAAGTTGAAGTCCATTTTGATTGATTTGACTGTGT
TATTTTGCGTG-3′.

The resulting strain was named BLT021.

pBOL34 was linearized by a Psil digestion and integrated in the ADH1 locus of BLT021. The transformants were grown on plates containing YCB (Difco) and 5 mM acetamide. For removal of the AmdS selection marker, colonies were inoculated in YEPD and grown for 6 hours in 2 ml tubes at 30° C. The cells were plated on YCB (Difco) and 40 mM fluoroacetamide and 0.1% ammonium sulphate.

Correct integration and recombination were confirmed by PCR. The correct integration of the fragment upstream was confirmed with the following primer set:

P5:
5′-GAACAATAGAGCGACCATGACCTTG-3′
and
P6:
5′-GACATCAGCGTCACCAGCCTTGATG-3′.

The correct integration of the fragment downstream was confirmed with the following primer set:

P7:
5′-GATTGAAGGTTTCAAGAACAGGTGATG-3′
and
P8:
5′-GGCGATCAGAGTTGAAAAAAAAATG-3′.

The resulting strain was named BLT057.

1. 2. Introduction ETFa, ETF13 and AcDH67 (lin1129) in BLT057

The electron transfer flavoproteins, ETFα[SEQ ID NO: 7], ETFβ [SEQ ID NO:9] and acetylating aldehyde dehydrogenase Listeria lnnocua lin1129 (here called Acdh67) [SEQ ID NO: 11] were codon pair optimized for S. cerevisiae as described in WO2008/000632 and expressed from yeast promoters and terminators as listed in Table 2.

TABLE 2
Promoters and terminators used for expression of codon pair optimized
(CpO) ETF genes and Acdh67 gene in S. cerevisiae

| | Promotor | Terminator |
|---|---|---|
| Etfα(CpO) SEQ ID NO: 8 | tef1 | tdh2 |
| Etfβ(CpO) SEQ ID NO: 10 | tdh2 | tef1 |
| Acdh67 (lin1129 Ec) | tdh3 | Adh |
| SEQ ID NO: 12 | | |

The integration vector expressing ETFα, ETFβ and Acdh67 (pBOL120, [SEQ ID NO: 15]) were synthesized by Geneart AG.

The vector was linearized with Stul and integrated in the ura3-52 locus of strain BLT057.

The transformants were grown in YNB (Difco) w/o amino acids+2% galactose to select for uracil prototrophic strains. The strains derived from strain BLT057 with pBOL120 integrated in the genome was designated strain: BLT075.

### 1.3. Nitrosoquanidine Mutagenesis (NTG) Mutagenesis

Strain BLT075 is inoculated from glycerol stock in 25 ml shake flask with Verduyn medium (Verduyn et al., 1992, Yeast 8:501-517)+4% galactose. At OD₆₀₀˜1, the culture was spun down.

The pellet was resuspended with 15 ml sterile MilliQ and tris maleate buffer.

Next, 0.025, 0.05 or 0.1 mg/ml NTG was added and incubated for 45 minutes at 25° C. in a shaking water bath. The mutagenesis was stopped by adding Na₂S₂O₃.5H₂O (1.67% w/v final concentration). The cells were spun down and washed with physiological salt. The mutant batches were plated and incubated at 30° C. to determine survival rates.

Mutants selected were from batches with a survival rate between 14 and 73%.

A few thousand colonies were screened for butanol production in 24 well plates containing 4 ml Verduyn medium and 4% (w/v) galactose. After 72 hours cultivation at 30° C., the 24 well plates were spun down and butanol concentration in the supernatant was determined by GC analysis.

The top 500 was selected for further testing in shake flasks. One hundred ml shake flasks containing 50 ml Verduyn medium with 4% galactose were inoculated with 0.5 ml culture from 24 wells plates precultures. The shake flasks were grown for 72 hours in an Infors shaker at 180 rpm and 30° C. The cultures were spun down and the butanol and ethanol concentrations were determined in the supernatant by GC as described below.

Mutant Saccharomyces cerevisiae BLT196 and BLT189 were selected for increased butanol production (Table 3). These mutants were deposited at the CBS (Centraalbureau voor Schimmelcultures, P.O. Box 85167, 3508 AD UTRECHT, The Netherlands) on Apr. 25, 2008, and Jun. 16, 2008, under the terms of the Budapest Treaty under accession number CBS 122885 and CBS 123039, respectively.

TABLE 3
Yield of butanol on sugar (Ybs) in gram per gram. Yield of ethanol on
sugar (Yes) and the butanol:ethanol ratio.

| Strain | Ybs (g/g) | Yes (g/g) | Ratio |
|---|---|---|---|
| BLT196 | 0.026 | 0.257 | 1:10 |
| CBS 122885 | | | |
| BLT189 | 0.023 | 0.258 | 1:11 |
| CBS 123039 | | | |
| BLT075 | 0.017 | 0.211 | 1:12 |
| CEN.PK113-7D | 0 | 0.34 | — |

This example shows that mutagenesis increased the butanol production in a butanol producing yeast.

### 1.4. GC Analysis

The butanol concentration was determined in the supernatant of the culture. Samples were analysed on a HS-GC equipped with a flame ionisation detector and an automatic injection system. Column J&W DB-1 length 30 m, id 0.53 mm, df 5 μm. The following conditions were used: helium as carrier gas with a flow rate of 5 ml/min. Column temperature was set at 110° C. The injector was set at 140° C. and the detector performed at 300° C. The data was obtained using Chromeleon software. Samples were heated at 60° C. for 20 min in the headspace sampler. One (1) ml of the headspace volatiles were automatically injected on the column

### Example 2

### Modification of an Existing Ethanol Distillation System to a System Capable of Recovering Both Ethanol and Butanol

Process modeling package Aspen Plus 2006.5 was used to simulate the distillation section of an existing ethanol plant and to calculate the adaptations needed to convert this plant into an ethanol/butanol plant.

### 2.1. Simulation of an Existing Ethanol Plant for Ethanol Production

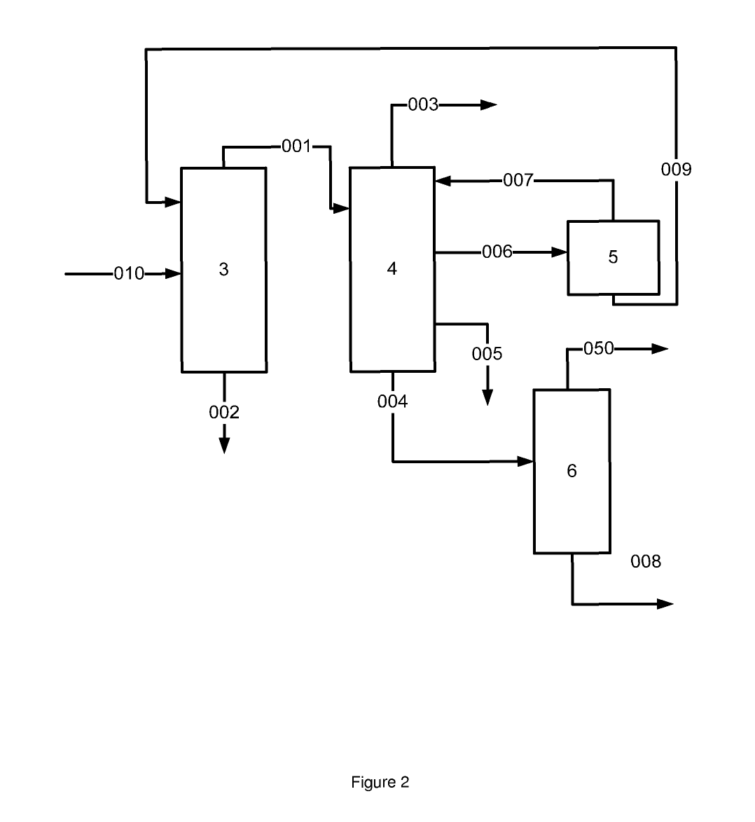

An existing ethanol plant was simulated wherein only ethanol is produced. In that case, only column 3 and 4 of FIG. 2 are used. Ethanol was recovered from a stripper column 3, wherein fermentation broth 010 with approximately 7.3 wt % ethanol is fed to the top of the column after exchanging heat with the bottom stream of the stripper column 3. Via direct steam injection ethanol was stripped form the broth resulting in a vapor stream 001 comprising 63.5 wt % ethanol. The liquid stream 002 leaving the bottom of the stripper column comprises 0.01 wt % ethanol. The ethanol rich vapor was fed to a column 4 wherein via refluxing the ethanol was concentrated to 93.2 wt % ethanol at the top of the column. Water leaving the bottom of column 4 comprises less then 0.01 wt % ethanol. Heat was supplied to the column via direct steam injection. The steam used for both columns was 130° C.

### 2.2. Simulation of an Existing Ethanol Plant, for Ethanol and Butanol Production

An existing ethanol plant was simulated, wherein a butanol and ethanol solution was produced with a ratio butanol:ethanol of between 1:2 to 1:100. Only column 3 and 4 of FIG. 2 were used. It was assumed that part of the ethanol in the feed was replaced by butanol, resulting in 6.3 wt % ethanol and 1 wt % butanol. This resulted in a vapor stream leaving the top of column 3 with 54.6 wt % ethanol and 8.7 wt % butanol. The water stream leaving the bottom of column 3 comprises less than 0.01 wt % ethanol and less than 0.01 wt % butanol. In column 4, both ethanol and butanol were concentrated at the top of the column and were harvested there. The ethanol and butanol concentrations were 66.8 wt % and 10.6 wt %, respectively, which means that there was 22.6 wt % of water left in this stream. The concentration factor achieved in column 4 was minimal.

### 2.3. Simulation of an Adapted Ethanol Plant, for Ethanol and Butanol Production

An adapted ethanol plant was simulated, wherein a butanol and ethanol solution was produced with a ratio butanol:ethanol of between 1:2 to 1:100, as indicated in FIG. 2. It was assumed that part of the ethanol in the feed was replaced by butanol, resulting in 6.3 wt % ethanol and 1 wt % butanol. This resulted in a vapor stream leaving the top of column 3 with 54.6 wt % ethanol and 8.7 wt % butanol. The water stream leaving the bottom of column 3 comprises less than 0.01 wt % ethanol and less than 0.01 wt % butanol.

By drawing a side stream from column 4 with 54.7 wt % butanol, 2.2 wt % ethanol and 43.1 wt % water and cooling this to 30° C., the natural tendency of phase separation was used to break the butanol-water azeotrope. The side stream mass flow is approximately 1.3 times that of the feed stream coming from column 3. A solvent rich phase 007 is fed back to column 4, one tray below the tray where the side stream is drawn from. The solvent rich phase consisted of 73.6 wt % butanol, 2.5 wt % ethanol and 23.9 wt % water. The water layer consisted of 89.3 wt % water, 9.2 wt % butanol and 1.5 wt % ethanol, and was fed to the column 3. By doing this, pure butanol can be harvested at the bottom of column 4 while still producing hydrous ethanol with 7 wt % water at the top of the rectifier. A reboiler was used to supply the heat required for column 4. The total heat demand per ton of solvent of this butanol-ethanol recovery system was similar to an ethanol recovery system. The gas flow in columns 3 and 4 for the recovery of butanol and ethanol was also comparable to the gas flow in columns 3 and 4 for the recovery of ethanol alone.

This example shows that minor adaptations (installation of a decanter and a reboiler) of an existing ethanol distillation set-up are required for the recovery of both ethanol and butanol from a process for the preparation of ethanol:butanol at a ratio 100:1 to 2:1

1. A eukaryotic cell capable of producing butanol and ethanol at a ratio butanol: ethanol of between 1:2 to 1:100.

2. A eukaryotic cell according to claim 1, wherein the cell comprises at least one inactivated nucleotide sequence encoding an enzyme that is required for the production of ethanol.

3. A eukaryotic cell according to claim 1, wherein the cell comprises a nucleotide sequence encoding a butyryl-CoA dehydrogenase and at least one nucleotide sequence encoding a heterologous electron transfer flavoprotein.

4. A eukaryotic cell according to claim 1, wherein the cell comprises a nucleotide sequence encoding a heterologous enzyme having enzymatic activity for converting pyruvate, acetaldehyde or acetate into acetyl-CoA in the cytosol.

5. A eukaryotic cell according to claim 1, wherein the cell is a yeast, preferably of the genus Saccharomyces.

6. A eukaryotic cell which is a Saccharomyces cerevisiae with accession number CBS 122885, or a Saccharomyces cerevisiae with accession number CBS 123039.

7. A process for increasing the butanol production of a eukaryotic cell capable of producing butanol comprising:
subjecting a population of eukaryotic cells capable of producing butanol to mutagenesis; and
selecting a population of mutant eukaryotic cells for increased butanol production.

8. Process for the preparation of butanol and ethanol comprising fermenting a eukaryotic cell according to claim 1, in a suitable fermentation broth, wherein butanol and ethanol are produced at a ratio butanol:ethanol of between 1:2 to 1:100.

9. Process according to claim 8, wherein the amount of butanol produced in the fermentation broth is at least 0.4 g/l.

10. Process according to claim 8, wherein the eukaryotic cell is a yeast, preferably belonging to the genus Saccharomyces.

11. Process according to claim 8, wherein butanol and ethanol are recovered from the fermentation broth.

12. Process according to claim 8, further comprising separating butanol from ethanol to obtain ethanol containing less than 1% butanol, and butanol containing less than 1% ethanol.

13. Process for the recovery of butanol and ethanol from an aqueous solution comprising butanol and ethanol wherein the ratio butanol:ethanol is between 1:2 to 1:100 comprising separating an ethanol/butanol/water mixture from the aqueous solution; separating an ethanol/water mixture from the ethanol/butanol/water mixture; separating a butanol/water mixture from the ethanol/butanol/water mixture; and recovering of butanol and ethanol.

14. Process according to claim 13 wherein the aqueous solution is a fermentation broth.

15. Process according to claim 13 wherein separating a butanol/water mixture comprises bringing part of the ethanol/butanol/water mixture to a temperature of between 10 to 40 degrees Celsius.

16. Process according to claim 1, which is carried out on an industrial scale.

17. A fermentation broth obtainable by a process according to claim 8, comprising butanol and ethanol at a ratio butanol:ethanol of between 1:2 to 1:100.

18. Use of butanol and/or ethanol obtainable by a process according to claim 8, as a chemical or as a biofuel.
